(19) United States
(12) Patent Application Publication
(10) Pub. No.: US 2007/0292407 A1
(43) Pub. Date: Dec. 20, 2007

(54) Methods of treatment for meconium aspiration syndrome

(75) Inventor: Vadim A. Ivanov, Hershey, PA (US)

Correspondence Address: Medlen & Carroll, LLP, Suite 350, 101 Howard Street, San Francisco, CA, 94105, US

(73) Assignee: The Board of Trustees Operating Michigan State University, East Lansing, MI (US)

(21) Appl. No.: 11/637,381

(22) Filed: Dec. 12, 2006

Related U.S. Application Data
(60) Provisional application No. 60/749,054, filed on Dec. 12, 2005.

(51) Int. Cl. A61K 31/395; A61K 38/47; A61K 38/55

(52) U.S. Cl. 424/94.61; 514/12; 514/537; 514/562

(57) ABSTRACT

The present invention relates to compositions and methods relating to prophylactic treatment and treatment of meconium aspiration syndrome comprising administering to a subject in need of treatment a therapeutically effective amount of acetylcysteine (N-acetyl-L-cysteine). One aspect of the invention relates to pharmaceutical compositions and methods of treatment comprising acetylcysteine and/or DNAse and/or a compound of the antiprotease family of drugs, administered singly or in combination, as medicament for treatment of meconium aspiration syndrome admixed with a pharmaceutically acceptable diluent, carrier, or excipient.

This application claims the benefit of U.S. Prov. Appl. 60/749,054, filed Dec. 12, 2005, herein incorporated by reference in its entirety.

### FIELD OF THE INVENTION

The present invention relates to compositions and methods relating to prophylactic treatment and treatment of meconium aspiration syndrome comprising administering to a subject in need of treatment a therapeutically effective amount of acetylcysteine (N-acetyl-L-cysteine). One aspect of the invention relates to pharmaceutical compositions and methods of treatment comprising acetylcysteine and/or DNAse and/or a compound of the antiprotease family of drugs, administered singly or in combination, as medicament for treatment of meconium aspiration syndrome admixed with a pharmaceutically acceptable diluent, carrier, or excipient.

### BACKGROUND OF THE INVENTION

Meconium aspiration syndrome (MAS) is a relatively common medical condition which describes severe lung injury with an associated significant morbidity in otherwise healthy and viable babies. There is no known association with a particular ethnic background, MAS affects both sexes equally and is exclusively a disease of newborns.

In the industrialized world, it is estimated that meconium in the amniotic fluid can be detected in 8-25% of all births after 34 weeks' gestation. Of those newborns with meconium-stained amniotic fluid, approximately 10% develop MAS. There are reports showing a decrease in the MAS incidence attributable to better obstetric practices such as expedited delivery of post-due babies [Gelfand et al., Clin Perinatol 2004; 31(3):445-52; herein incorporated by reference]. However a mortality rate estimate is that MAS is responsible for about 1000 deaths annually in the U.S. [Gelfand et al., Clin Perinatol 2004; 31(3):445-52; herein incorporated by reference].

Current prevention measures are either too difficult to implement, such as amnioinfusion or have questionable effectiveness such as oropharingeal suctioning before delivery of the shoulders or tracheal intubation and suctioning following delivery [Gelfand et al., Clin Perinatol 2004; 31 (3):445-52; Greenough et al., Eur J Pediatr 2005; 164(5): 329-30; all of which are herein incorporated by reference]. Further, infants showing symptoms of MAS are treated with ventilation techniques help to support altered respiratory function or with extracorporeal membrane oxygenation (ECMO). Thus a lack of an effective and reliable MAS prevention measure is further complicated by a virtual absence of effective treatment methods after MAS occurs in infants.

Therefore, a simple, effective and reliable MAS prevention treatment is needed in addition to effective MAS treatment methods to prevent fetal and infant mortality.

### SUMMARY OF THE INVENTION

The present invention relates to compositions and methods relating to prophylactic treatment of meconium aspiration syndrome comprising administering to a subject in need of treatment a therapeutically effective amount of a meconium enzyme inhibitor, specifically, acetylcysteine (N-acetyl-L-cysteine). One aspect of the invention relates to pharmaceutical compositions and methods of treatment comprising acetylcysteine and/or DNAse and/or a compound of the antiprotease family of drugs, administered singly or in combination, as medicament for treatment of meconium aspiration syndrome admixed with a pharmaceutically acceptable diluent, carrier, or excipient.

The present invention provides methods of treating meconium aspiration syndrome, comprising, a) providing, i) a pharmaceutical formulation of a meconium enzyme inhibitor compound and/or a meconium liquefying compound and/or a meconium antiinflammatory compound, wherein said compound is a therapeutically effective amount; and ii) a subject in need of prophylactic treatment for meconium aspiration syndrome, wherein said subject is pregnant or a fetus or an infant; and b) administering to a subject in need a therapeutically effective amount of a pharmaceutical formulation of a meconium enzyme inhibitor compound and/or a meconium liquefying compound and/or a meconium antiinflammatory compound for reducing symptoms of meconium aspiration syndrome in a fetus or infant. In one embodiment, the meconium enzyme inhibitor compound reduces activity of meconium enzymes or reduces activity of enzymes activated by contact with meconium. It is not intended that the present invention be limited to a particular meconium enzyme inhibitor compound and includes an antiprotease family of drugs, a buffer solution comprising a pH outside the optimal pH for pancreatic/intestinal digestive enzymes. In one embodiment, the meconium liquefying compound reduces viscosity and/or thickness of meconium and is selected from the group consisting of N-acetylcysteine and DNAse. In one embodiment, the meconium antiinflammatory compound is selected from a group consisting of antiprotease compounds. It is not intended that the present invention be limited to a particular antiprotease compound and includes Aprotinin (Trasylol); Gabexate mesilate (Foy); Camostate (Foy 305); Nafamostate mesilate (FUT 175); Sepimostate (FUT 187); Ulinostatin; E 3123; THL (lipase inhibitor); C1 INH (C1-esterase inhibitor); CaNa2 (phospholipase A2 inhibitor); Procaine (phospholipase A2 inhibitor); and Lexipafant (PAF inhibitor) ONO 3307. In one embodiment, the methods further comprise an artificial surfactant and administrating said surfactant. It is not intended that the therapeutic method of the present invention be limited to certain modes of administration. A variety of modes of administering the therapeutic composition are contemplated. In one embodiment, the therapeutic composition is administered by a mode selected from intratracheal, amnio-infusion, oral, instillation, lavage, spraying, vaporizing, misting, nebulizing, systemically, intravenously, and intramuscularly. In one embodiment, administering is oral with subsequent delivery of the active ingredients to the lungs or bloodstream for delivery of the active ingredient to the tracheal-bronchial tree. In one embodiment, reducing symptoms is reducing the severity of a symptom in the fetus or infant such as an airway obstruction, surfactant dysfunction, and chemical pneumonitis. In one embodiment, a subject is a human.

The present invention provides methods of treating meconium aspiration syndrome, comprising, a) providing, i) a pharmaceutical formulation of acetylcysteine, wherein said acetylcysteine is a therapeutically effective amount; and ii) a subject in need of prophylactic treatment for meconium aspiration syndrome; and b) administering to a subject in need a therapeutically effective amount of a pharmaceutical formulation of acetylcysteine. In one embodiment, a subject in need is a pregnant subject, wherein said pregnant subject is a maternal subject. In one embodiment, the pregnant subject is in labor. In one embodiment, the subject in need is a subject whose placental membrane may have ruptured or has ruptured. In one embodiment, the pregnant subject comprises a fetus. In one embodiment, the pregnant subject comprises a fetus at risk for meconium aspiration syndrome or showing symptoms of meconium aspiration syndrome. In one embodiment, a risk for meconium aspiration syndrome is a risk symptom selected from the group consisting of meconium-stained amniotic fluid, intrauterine distress, placental insufficiency, maternal hypertension, preeclampsia, oligohydramnios, decreased amniotic fluid volume, maternal drug abuse, maternal tobacco use, and maternal cocaine abuse. In one embodiment, a showing symptoms of meconium aspiration syndrome is showing a symptom such as meconium-stained amniotic fluid, airway obstruction, surfactant dysfunction, and chemical pneumonitis. In one embodiment, the subject is a fetus or an infant. In one embodiment, a fetus or infant is at risk for meconium aspiration syndrome or showing symptoms of meconium aspiration syndrome. In one embodiment, a showing symptoms of meconium aspiration syndrome is showing a symptom selected from the group consisting of meconium-stained amniotic fluid, airway obstruction, surfactant dysfunction, and chemical pneumonitis. In one embodiment, the subject is a human subject. In one embodiment, a therapeutically effective amount reduces the toxicity of meconium. In one embodiment, the methods further comprise administering a therapeutically effective amount of DNAse or an antiprotease compound. In one embodiment, a DNAse is human deoxyribonuclease I (rhDNase) or Pulmozyme® (dornase alfa). In one embodiment, an antiprotease compound is selected from the group consisting of Aprotinin (Trasylol); Gabexate mesilate (Foy); Camostate (Foy 305); Nafamostate mesilate (FUT 175); Sepimostate (FUT 187); Ulinostatin; E 3123; THL (lipase inhibitor); C1 INH (C1-esterase inhibitor); CaNa2 (phospholipase A2 inhibitor); Procaine (phospholipase A2 inhibitor); and Lexipafant (PAF inhibitor) ONO 3307. In one embodiment, an acetylcysteine is administered consecutively, simultaneously, or sequentially with a meconium enzyme inhibitor compound and/or a meconium liquefying compound and/or a meconium antiinflammatory compound and/or a surfactant. In one embodiment, an acetylcysteine and a meconium antiinflammatory compound are administered simultaneously. It is not intended that the therapeutic method of the present invention be limited to certain modes of administration. A variety of modes of administering the therapeutic composition are contemplated. In one embodiment, the therapeutic composition is administered by a mode selected from intratracheal, amnio-infusion, oral, instillation, lavage, spraying, vaporizing, misting, nebulizing, systemically, intravenously, and intramuscularly. In one embodiment, administering is oral with subsequent delivery of the active ingredients to the lungs or bloodstream for delivery of the active ingredient to the tracheal-bronchial tree. In one embodiment, the methods further comprise administering a therapeutically effective amount of a surfactant. In one embodiment, the acetylcysteine is administered consecutively, simultaneously, or sequentially with a meconium enzyme inhibitor compound and/or a meconium liquefying compound and/or a meconium antiinflammatory compound and/or a surfactant. In one embodiment, the acetylcysteine and a meconium antiinflammatory compound are administered simultaneously.

The present invention provides methods of treating an infant presenting with symptoms of meconium aspiration syndrome comprising administering to the trachea of the infant a therapeutically effective amount of acetylcysteine and a surfactant.

In further embodiments, the present invention provides a pharmaceutical preparation comprising therapeutically effective amounts of a meconium liquefying component and at least one antiprotease compound. The present invention is not limited to the use of any particular meconium liquefying component. In some embodiments, the meconium liquefying component is selected from the group consisting of DNase and N-Acetylcysteine or a combination thereof. The present invention is not limited to the use of any particular antiprotease (protease inhibitor) compound. Suitable antiprotease compounds include, but are not limited to, Aprotinin (Trasylol), Gabexate mesilate (Foy), Camostate (Foy 305), Nafamostate mesilate (FUT 175), Sepimostate (FUT 187), Ulinostatin, E 3123, THL (lipase inhibitor), ONO5046 (PMN-elastase inhibitor), C1 INH (C1-esterase inhibitor), AT3 (antithrombin III inhibitor), CaNa2 (phospholipase A2 inhibitor), Procaine (phospholipase A2 inhibitor), Lexipafant (PAF inhibitor) ONO 3307. In some embodiments, the preparation is buffered to a pH outside the optimal pH for pancreatic/intestinal digestive enzymes. In some preferred embodiments, an acidic buffer and/or acidifier are included in pharmaceutical preparation. In some further embodiments, the preparation comprises a pharmacologically effective diluent. The present invention is not limited to any particular pharmaceutical preparation. In some embodiments, the preparation is a liquid that is formulated to be deliverable as a spray, vapor or mist.

In further embodiments, the present invention provides kits for therapeutic use comprising therapeutically effective amounts of a first component, wherein said first component is meconium liquefying substance and a second component, wherein said second component is at least one antiprotease compound, wherein said first and second components are provided in therapeutically effective amounts. The present invention is not limited to the use of any particular meconium liquefying component. In some embodiments, the meconium liquefying component is selected from the group consisting of DNase and N-Acetylcysteine or a combination thereof. The present invention is not limited to the use of any particular antiprotease (protease inhibitor) compound. Suitable antiprotease compounds include, but are not limited to, Aprotinin (Trasylol), Gabexate mesilate (Foy), Camostate (Foy 305), Nafamostate mesilate (FUT 175), Sepimostate (FUT 187), Ulinostatin, E 3123, THL (lipase inhibitor), ONO5046 (PMN-elastase inhibitor), C1 INH (C1-esterase inhibitor), AT3 (antithrombin III inhibitor), CaNa2 (phospholipase A2 inhibitor), Procaine (phospholipase A2 inhibitor), Lexipafant (PAF inhibitor) ONO 3307. In some embodiments, the first and second components are buffered to a pH outside the optimal pH for pancreatic/intestinal digestive enzymes. In some embodiments, the kits further comprise a third component that is a buffer solution with pH outside the optimal pH for pancreatic/intestinal digestive enzymes. In some embodiments, the first and second components are provided in a pharmacologically effective diluent. In some embodiments, the first and second components are provided as liquids that are formulated to be deliverable as a spray, vapor or mist. In some embodiments, the first, second and third components are provided in separate containers. In other embodiments, the components are provided in the same container. In some embodiments, the third component also acts as a surfactant. In some embodiments, the kits further comprise instructions for the prophylactic or therapeutic treatment of meconium aspiration.

Definitions

To facilitate an understanding of the present invention, a number of terms and phrases are defined below:

As used herein, the term “subject” refers to a mother or fetus or infant.

The terms, “mother” or “maternal subject” or “pregnant subject” refers to a biological parent of an infant or comprising a fetus attached by an umbilical cord.

The terms, “fetus” or “fetal subject” refers to a developing mammal after the embryonic stage and before birth. In other words, a fetus is a developing mammal attached to a mother by an umbilical cord.

The term “subject” refers to a patient, such as a pregnant patient or a fetus or an infant. A subject is a mammal, such as a human.

The term, “umbilical cord” is a tube that connects a developing embryo or fetus to its placenta.

The terms, “infant” or “infant subject” refers to a very young mammal, such as a human child, that is not attached to a biological mother by an umbilical cord and includes any child under the age of legal adulthood. A human infant less than a month old is referred to as a “newborn infant” or a “neonate” or “newborn” or “baby.” The term “newborn” includes premature infants and postmature infants, as well as full term newborns.

The terms, “neonatology” or newborn care” or “neonatal care” refers to care of an infant commonly obtained in the neonatal or nursery unit of a hospital.

The terms, “amniotic sac rupture” or “water breaks” or “when a doctor ruptures the amniotic membranes” refers to a release of amniotic fluid from the amniotic sac.

The terms, “meconium” or “fetal plug” refers to a first intestinal discharge from a newborn that is a viscous, dark green substance composed of intestinal epithelial cells, lanugo, mucus, and intestinal secretions, such as bile and pancreatic juice. Meconium comprises solids and fluids, wherein solids comprise intestinal secretions, mucosal cells, and solid elements of swallowed amniotic fluid, and wherein water is the major liquid constituent, typically comprising 85-95% of meconium.

The terms, “meconium aspiration syndrome” or “MAS” refer to a relatively common medical condition that describes severe lung injury with significant morbidity in otherwise healthy and viable babies. MAS describes a group of symptoms of which a MAS victim my present with at least one or more of meconium-stained amniotic fluid, airway obstruction, surfactant dysfunction, and chemical pneumonitis, as in “showing a symptom of MAS.”

The terms, “fetus at risk” or “infant at risk” or “at risk” in reference to meconium aspiration syndrome or showing symptoms of meconium aspiration syndrome refers to a MAS risk symptom such as meconium-stained amniotic fluid, intrauterine distress, placental insufficiency, maternal subject's hypertension, preeclampsia, oligohydramnios, decreased amniotic fluid volume, maternal subject's drug abuse, maternal subject's tobacco use, and maternal subject's cocaine abuse.

The term, “symptoms of meconium aspiration syndrome” refers to any symptoms characteristic of a subject with meconium aspiration syndrome.

The term, “reducing symptoms of meconium aspiration syndrome” refers to a qualitative or quantitative reduction in detectable symptoms, including but not limited to a detectable impact on the rate of recovery from disease; examples of symptoms include but are not limited to airway obstruction, surfactant dysfunction, and chemical pneumonitis.

The term, “reducing symptoms of meconium aspiration syndrome in a fetus or infant” refers to a reduction or lessening of the severity of or clinical presentation of a symptom of MAS, such as reducing the severity of airway obstruction, surfactant dysfunction, and chemical pneumonitis.

The terms, “therapeutically effective amount” or “therapeutically effective” refer to an amount of a compound or pharmaceutical formulation that reduces or prevents the toxicity of meconium symptom or reduces the severity of a MAS symptom.

The term “meconium enzyme inhibitor compound” refers to a compound that reduces activity of meconium enzymes or reduces activity of enzymes activated by contact with meconium for example, antiprotease family of drugs, a buffer solution comprising a pH outside the optimal pH for pancreatic/intestinal digestive enzymes.

The term “meconium liquefying compound” refers to a compound that reduces viscosity and/or thickness of meconium, such as N-acetylcysteine and recombinant DNAse

The term “meconium antiinflammatory compound” refers to a compound that reduces inflammatory reactions associated with MAS, such as antiprotease compounds.

The term “administering” refers to contacting a subject with a compound by a variety of modes such as intratracheal, amnioinfusion, oral, instillation, lavage, spraying, vaporizing, misting, nebulizing, systemically, intravenously, and intramuscularly. For the purposes of the present inventions, administering in reference to “oral” administering further refers to subsequent delivery of the active ingredients to the lungs or bloodstream for delivery of the active ingredient to the tracheal-bronchial tree

The term “artificial surfactant” refers to a synthetic compound that mimics or compensates for natural surfactant activity.

The terms “pharmaceutical formulation” and “pharmaceutical preparation” refer to a pharmaceutical compound in a formulation that is administered to a subject.

The term “prophylactic treatment” refers to any medical or public health procedure whose purpose is to prevent and/or treat a disease or syndrome. Prophylactic treatments are divided between primary prophylaxis (to prevent the development of a disease) and secondary prophylaxis (whereby the disease has already developed and the patient is protected against worsening of this process).

The terms, “ball-valve effect” or “ball-valve obstruction” “ball-valve” refer to a type of airway obstruction related to induction of respiratory symptoms and/or pneumothorax. An example of a ball-valve effect is an effect of a solid obstruction or foreign body that leads to early air trapping and overinflation of the bronchial area and/or lungs. As a further example, an airway foreign body has been a major cause of morbidity and mortality in the United States.

The present invention contemplates the use of both atomizers and nebulizers of various types. An “atomizer is an aerosol generator without a baffle, whereas a “nebulizer” uses a baffle to produce smaller particles.

The present invention contemplates in some embodiments utilizing nebulizers and aerosol drug delivery devices based upon piezo electronic technology (e.g. Pari GmBh (Starnberg, Germany) e-Flow™ electronic nebulizers based on piezo ceramic electronic transducers), including portable nebulizers and aerosol devices (e.g. Omron Healthcare, Inc Portable Ultrasonic Nebulizer, NE-U03V MicroAir) and inhaled drug delivery technology (e.g. Mystic™ drug inhalation technology BattellePharma).

The term “Acute Respiratory distress syndrome” or “ARDS” refers to a sudden, life threatening lung failure from inflamed alveoli that fill with liquid. It is often treated by mechanical ventilation with antibiotics.

The terms “compound” or “pharmaceutical compound” refer to any chemical entity, pharmaceutical, drug, and the like that can be used to treat or prevent a disease, illness, sickness, or disorder of bodily function. Test compounds or compounds comprise both known and potential therapeutic compounds. A test compound can be determined to be therapeutic by screening using the screening methods of the present invention.

The term “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Biological samples may be animal, including, human, meconium, fluid (e.g., blood, plasma and serum), solid (e.g., stool), and tissue (e.g., tissues of the gut or central nervous system).

### DETAILED DESCRIPTION OF THE INVENTION

The present invention relates to compositions and methods relating to prophylactic treatment and treatment of meconium aspiration syndrome comprising administering to a subject in need of treatment a therapeutically effective amount of acetylcysteine (N-acetyl-L-cysteine). One aspect of the invention relates to pharmaceutical compositions and methods of treatment comprising acetylcysteine and/or DNAse and/or a compound of the antiprotease family of drugs, administered singly or in combination, as medicament for treatment of meconium aspiration syndrome admixed with a pharmaceutically acceptable diluent, carrier, or excipient.

Presently, modern medicine does not offer disease-modifying treatment for meconium aspiration syndrome (MAS). The inventor contemplates that several medications with established safety profiles employed for similar or other medical conditions would be useful for treating MAS. For example, N-acetylcysteine and DNAse have the capability to reduce viscosity and thickness of meconium material by breaking disulfide bonds and slicing DNA, respectively. Further, N-acetylcysteine, antiprotease drugs, or low pH buffer solutions may have the capability to neutralize meconium's digestive enzymes responsible for lung damage in patients with MAS. Thus these compounds have great potential to reduce meconium's pathogenic properties which in turn could alleviate MAS severity.

Thus the inventor contemplates methods of treatment for meconium aspiration syndrome (MAS). The method could be used for treatment in victims of MAS and/or MAS prophylaxis in fetuses at risk of contracting MAS. A theory of pathophysiology describes meconium as exerting its pathological action in the airways by mechanical obstruction and by causing a ball-valve effect plus via chemical damage to surfactant and lung tissues. Therapeutic modalities currently used for MAS treatment aim mainly at simple substitution of an affected function, such as administrating artificial surfactant. A positive effect is observed when surfactant inactivated by contact with meconium is replenished by an artificial one. Further, different ventilation techniques help to support altered respiratory function or to substitute it with extracorporeal membrane oxygenation (ECMO). However, prevention measures offered previously are either too difficult to implement such as by amnioinfusion or have questionable effectiveness like oropharingeal suctioning before delivery of the shoulders or tracheal intubation and suctioning to physically remove meconium.

The inventor contemplates compositions and methods for inhibiting and/or preventing meconium from exerting its pathological properties either prior to aspiration from amniotic fluid and/or after aspiration into the infant's tracheal-bronchial tree. The invention comprises using medications which are capable of changing meconium's physical properties, mainly by liquefying it, and further by using compounds capable of neutralizing its chemical aggressiveness toward the lung and tracheal-bronchial tree.

The invention contemplates the use of compositions and treatments comprising the following compounds for treatment and/or prevention of the MAS: a meconium enzyme inhibitor compound and/or a meconium liquefying compound and/or a meconium antiinflammatory compound, wherein said compound(s) are provided in a therapeutically effective amount. Examples of a meconium enzyme inhibitor compound include, but are not limited to, acetylcysteine (N-acetyl-L-cysteine), a buffer solution with pH outside the optimal pH for pancreatic/intestinal digestive enzymes which thus is capable of stopping/reducing the activity of the destructive enzymes of meconium, an antiprotease compound selected from an antiprotease family of drugs, et cetera. Examples of antiprotease family of drugs, which are capable of stopping/reducing the activity of the destructive enzymes of meconium and reduce the body's inflammatory response to the presence of meconium include, but are not limited to: Aprotinin (Trasylol); Gabexate mesilate (Foy); Camostate (Foy 305); Nafamostate mesilate (FUT 175); Sepimostate (FUT 187); Ulinostatin; E 3123; THL (lipase inhibitor); C1 INH (C1-esterase inhibitor); CaNa2 (phospholipase A2 inhibitor); Procaine (phospholipase A2 inhibitor); and Lexipafant (PAF inhibitor) ONO 3307. Examples of a meconium liquefying compound include, but are not limited to, acetylcysteine (N-acetyl-L-cysteine), DNAse (Dornase Alfa; Pulmozyme®), et cetera.

The above mentioned medications are administered to a subject in need in a pharmacologically acceptable concentration in a pharmacologically acceptable diluent separately, or mixed together. The medication are delivered locally to the tracheal-bronchial tree; by instillation or lavage; inhalation of sprayed, vaporized or mist preparation of the medication; or systemically, for example, intravenously, intramuscular or via mouth with subsequent delivery of the active ingredients the to lungs and tracheal-bronchial tree via bloodstream.

Further, the invention encompasses an MAS prophylactic measure, one or several of the above mentioned medications could be used to neutralize or liquefy meconium prior to it's aspiration in a clinical situation when there is a risk of meconium aspiration. In one embodiment, the medication would be used for amnioinfusion or any other similar treatment method, thus neutralizing meconium which is already present in the amniotic sac or meconium present elsewhere prior to its aspiration by a fetus or infant; see, for examples, Inanov, 2006, Med Hypotheses, 66(4):808-10. Epub 2005 Dec. 20; herein incorporated by reference in its entirety.

I. Current Treatment Modalities.

One theory is that meconium exerts its pathological action in the airways of a fetus or infant by mechanical obstruction and/or by causing a ball-valve effect plus via chemical damage to surfactant and lung tissues [Gelfand et al., Clin Perinatol 2004; 31(3):445-52; herein incorporated by reference]. The therapeutic modalities which are currently used for MAS treatment aim mainly at simple substitution of an affected function. Current treatment compositions shows some positive effect, such as when surfactant inactivated by contact with meconium is replenished by an artificial one [Soll et al., Cochrane Database Syst Rev 2000:CD002054; herein incorporated by reference] or by administering an albumin compound [U.S. Pat. No. 6,838,441; herein incorporated by reference]. Different ventilation techniques help to support altered respiratory function or to substitute it with ECMO [Gelfand et al., Clin Perinatol 2004; 31(3):445-52; Soll et al., Cochrane Database Syst Rev 2000:CD002054; all of which are herein incorporated by reference]. However, there is a lack of published studies aiming at removal or/and neutralizing meconium—the causative agent of these types of medical problems.

The same skewed medical picture is seen in bench studies of MAS. Current studies are concentrated on the investigation of consequences of meconium action toward lungs and airways [Gelfand et al., Clin Perinatol 2004; 31(3):445-52; herein incorporated by reference]. There is a lack of understanding which components of meconium are responsible for its pathological effects and properties. The apparent lack of attention to the composition of meconium and its mechanisms of action impedes progress in the development of new MAS treatment modalities.

II. MAS Treatments.

There is a lack of published information on the underlying mechanisms of meconium pathogenicity in MAS. Published information describes brief theoretical investigation of MAS that provides potential causes of symptoms based upon physical properties of meconium.

Meconium staining of amniotic fluid occurs in 11-22% of all deliveries. Meconium aspiration syndrome occurs in approximately 2% of these deliveries. Release of meconium into the amniotic fluid is usually the result of in utero hypoxia and/or fetal distress. When meconium is passed more than 4 hours before delivery, the infant's skin will be meconium stained. The effects of meconium in amniotic fluid are well documented. Meconium directly alters the amniotic fluid, reducing antibacterial activity and subsequently increasing the risk of perinatal bacterial infection. Additionally, meconium is irritating to fetal skin, thus increasing the incidence of erythema toxicum. However, the most severe complication of meconium passage in utero is aspiration of stained amniotic fluid before, during, and after birth. Aspiration induces hypoxia via 3 major pulmonary effects, which are airway obstruction, surfactant dysfunction, and chemical pneumonitis.

Intrauterine distress in utero causes meconium passage into the amniotic fluid. Factors that promote the passage in utero include placental insufficiency, maternal hypertension, preeclampsia, oligohydramnios, and maternal drug abuse, especially of tobacco and cocaine. Meconium-stained amniotic fluid may be aspirated during labor and delivery, causing neonatal respiratory distress. Because meconium is rarely found in the amniotic fluid prior to 34 weeks' gestation, meconium aspiration chiefly affects infants at term and postterm.

Amniotic sac rupture and fluid release is when the water breaks or the doctor ruptures the aminotic membranes, the membranes are stained a brownish or yellowish color; this is meconium in the amniotic fluid. In other words, the fetus has had a bowel movement. This most often occurs if the woman is past due or because of fetal distress.

Pathophysiology in utero is meconium passage results from neural stimulation of a mature GI tract and usually results from fetal hypoxic stress. As the fetus approaches term, the GI tract matures, and vagal stimulation from head or cord compression may cause peristalsis and relaxation of the rectal sphincter leading to meconium passage.

A meconium distressed fetus will make reflex gasping movements and aspirate meconium stained fluid into the tracheal-bronchial tree. After the first breath, the infant will deposit the aspirated meconium stained fluid further down the bronchial tree and subsequently cause a mechanical blockage of alveoli and small airways with a resultant ball-valve type obstruction.

Airway obstruction is partial or complete obstruction of the airways by meconium results in atelectasis. Partial obstruction causes air trapping and hyperdistention of the alveoli, commonly termed the ball-valve effect. Hyperdistention of the alveoli occurs from airway expansion during inhalation and airway collapse around inspissated meconium in the airway, causing increased resistance during exhalation. The gas that is trapped, hyperinflating the lung, may rupture into the pleura (pneumothorax), mediastinum (pneumomediastinum), or pericardium (pneumopericardium).

An infant born via breech presentation often passes meconium prior to delivery, and further may pass meconium without showing signs of fetal distress (see, Holtzman R. B., et al. Perinatal management of meconium staining of the anmiotic fluid. Clin Perinatol, 1989; 16:825-838 and Klein, Iowa Neonatology Handbook: Pulmonary Care of the Infant with the Meconium Aspiration Syndrome; herein incorporated by reference in their entirety).

Surfactant dysfunction is where several constituents of meconium, especially the free fatty acids (e.g., palrnitic, stearic, oleic), have a higher minimal surface tension than surfactant and strip it from the alveolar surface, resulting in diffuse atelectasis.

Chemical pneumonitis is where enzymes, bile salts, and fats in meconium irritate the airways and parenchyma, causing a release of cytokines and resulting in a diffuse pneumonia that may begin within a few hours of aspiration. These pulmonary effects can produce gross ventilation-perfusion (V-Q) mismatch. To complicate matters further, many infants with meconium aspiration syndrome (MAS) have primary or secondary persistent pulmonary hypertension of the newborn (PPHN) as a result of chronic in utero stress and thickening of the pulmonary vessels. Finally, though meconium is sterile, its presence in the air passages can predispose the infant to pulmonary infection.

In some embodiments, the present invention provides pharmaceutical compounds and formulations that alter meconium and modify the course of the MAS. In some embodiments, at least two or more approaches for novel treatments for MAS are utilized. In one aspect of the invention, the invention contemplates altering meconium's physical properties, mainly by liquefying it. This provides a benefit by producing a less viscous and less sticky meconium that is easier to mobilize and remove from the tracheal-bronchial tree. Even if the meconium stays in the lungs, the liquefied meconium would be less capable to cause mechanical obstruction and a ball-valve effect in the airways. In another aspect of the invention, proteases contained in the meconium that cause adverse effects are inhibited. The proteases may be inhibited via protease inhibitors, compounds that breakdown proteins, or by creating conditions under which common proteases do not function efficiently.

Accordingly, in some embodiments, the pharmaceutical formulations of the present invention are utilized to neutralize meconium, which is a mixture of products swallowed by the fetus (amniotic fluid) and gastrointestinal secretions [Antonowicz et al., Adv Pediatr 1979; 26:275-310; herein incorporated by reference]. Further, meconium has a high protein content [Antonowicz et al., Adv Pediatr 1979; 26:275-310; herein incorporated by reference]. Accordingly, in some preferred embodiments, the compositions and kits of the present invention comprise one or more of the following components:

1) a therapeutically effective amount of a DNase, such as an inhaled human recombinant DNase [Shak et al., Proc Natl Acad Sci USA 1990; 87:9188-92; herein incorporated by reference].

2) a therapeutically effective amount of a compound that aids in the breakdown of proteins such as N-acetylcysteine [Mosby's Drug Consult Copyright c 2005 Mosby Inc.; herein incorporated by reference] [Burke et al., J Pediatr Surg 2002; 37(5):760-4; herein incorporated by reference];

3) a therapeutically effective amount of compound that neutralizes the chemical aggressiveness of meconium such as an antiprotease compound [Gelfand et al., Clin Perinatol 2004; 31(3):445-52; herein incorporated by reference], [Antonowicz et al., Adv Pediatr 1979; 26:275-310; Harries et al., 1978; 34(1):75-8; herein incorporated by reference].

The compounds described above may be used alone or in combination. In some embodiments, the pharmaceutical formulations are provided in a kit. In some embodiments, the pharmaceutical formulations are provided in the same container. In other embodiments, the pharmaceutical formulation are provided in separate containers. In either event, the compounds described above are provided in a therapeutically effective amount. A therapeutically effective amount is that amount of the compound that produces a desired therapeutic effect in vivo. Examples of desired therapeutic effects for the present invention include, but are not limited to, liquefication of meconium and inhibition of inflammation caused by ingestion of meconium.

In some embodiments, the treatments of the present invention utilize a broad-spectrum antiprotease lavage to inactivate the digestive enzymes. The majority of digestive enzymes originate from the pancreas and the intestine [Antonowicz et al., Adv Pediatr 1979; 26:275-310; herein incorporated by reference] and require neutral or slightly alkaline pH to exhibit digestive/destruction action [Guyton et al., 2000, ISBN 0-7216-8677-X. p. 748; herein incorporated by reference]. Thus, the kits and pharmaceutical preparations of the present invention for use in treating and/or preventing MAS may further comprise an effective amount of a buffer or acidifier, wherein following administration of the buffer or acidifier digestive enzymatic activity is inhibited. In some preferred embodiments, administration of the buffer causes slight acidification or just neutralization of the pH in the tracheo-bronchial tree. In some embodiments, the acidic buffer is phosphate or potassium dihydrogen phosphate. In further embodiments, the acidifier is aspartic acid, glutamic acid, folic acid or citric acid. The present invention is not limited to the use of any particular buffer or acidifier. Furthermore, in some embodiments, both a buffer and acidifier are utilized in the pharmaceutical formulations or kits.

Various compounds that may be included in the pharmaceutical formulation of the present invention are described in detail below.

A. N-Acetylcysteine Treatments for MAS.

N-Acetylcysteine shows an ability to alter meconium's physical properties. Since N-acetylcysteine's main mode of action is via altering protein structure [Shak et al., Proc Natl Acad Sci USA 1990; 87:9188-92; herein incorporated by reference], a composition comprising N-Acetylcysteine provides an additional benefit of inactivating digestive enzymes. In particular, in some preferred embodiments, an N-Acetylcysteine compound is used for treating and/or preventing MAS, for example, by reducing meconium viscosity. An N-Acetylcysteine compound was shown to have the ability for significant reduction of meconium viscosity in vitro [Burke et al., J Pediatr Surg 2002; 37(5):760-4; herein incorporated by reference]. In contrast, the present invention contemplates the use of an N-Acetylcysteine compound in vivo. In some embodiments, the use of a pharmaceutical formulation of an N-Acetylcysteine compound provides the additional benefit of neutralizing digestive enzymes in meconium. Thus, N-acetylcysteine is an ideal treatment for in vivo treatment of MAS and provides a dual action of breaking down proteins for reducing meconium viscosity and increasing chemical aggressiveness for inactivating digestive enzymes in and/or triggered by meconium.

Accordingly, in some preferred embodiments, the present invention provides a pharmaceutical formulation for treating MAS that comprises a therapeutically effective amount of N-Acetylcysteine. In some embodiments, the pharmaceutical formulation further comprises other components described herein, including but not limited to one or more of the following: and acidifier, a buffer, a protease inhibitor, a DNase, and a surfactant. In other embodiments, the components are included in two or more separate containers in a kit.

B. DNAse Treatments for MAS.

In some embodiments, the present invention contemplates the use of DNAse compositions for liquefying meconium, for example, a DNAse, a recombinant DNAse and the like. Accordingly, in some preferred embodiments, the present invention provides a pharmaceutical formulation for treating MAS that comprises a therapeutically effective amount of DNase. In some embodiments, the pharmaceutical formulation further comprises other components described herein, including but not limited to one or more of the following: and acidifier, a buffer, a protease inhibitor, N-acetylcysteine, and a surfactant. In other embodiments, the components are included in two or more separate containers in a kit.

C. Antiprotease Treatments for MAS.

Broad-spectrum antiproteases as pharmaceutical formulations are contemplated for treating MAS by neutralization of meconium's digestive enzymes and/or enzymes released in vivo by contact with meconium. The antiproteases may be used alone or in combination with N-Acetylcysteine and/or DNase. Examples of suitable antiproteases include, but are not limited to, Aprotinin (Trasylol), Gabexate mesilate (Foy), Camostate (Foy 305), Nafamostate mesilate (FUT 175), Sepimostate (FUT 187), Ulinostatin, E 3123, THL (lipase inhibitor), ONO5046 (PMN-elastase inhibitor), C1 INH (C1-esterase inhibitor), AT3 (antithrombin III inhibitor), CaNa2 (phospholipase A2 inhibitor), Procaine (phospholipase A2 inhibitor), Lexipafant (PAF inhibitor) ONO 3307. It is contemplated that the antiprotease alleviates the chemical damage to lungs by aspirated meconium. Accordingly, in some preferred embodiments, the present invention provides a pharmaceutical formulation for treating MAS that comprises a therapeutically effective amount of protease inhibitor. In some embodiments, the pharmaceutical formulation further comprises other components described herein, including but not limited to one or more of the following: and acidifier, a buffer, a DNase, N-acetylcysteine, and a surfactant. In other embodiments, the components are included in two or more separate containers in a kit.

D. Surfactant and/or low pH Buffer Solution for Treatments of MAS.

One known medicine which is capable of altering MAS course is surfactant. The pH of a pharmaceutical formulation of surfactant is in the range 5.0-6.2 [surfactant; Mosby's Drug Consult Copyright c 2005 Mosby Inc.; herein incorporated by reference]. Thus the inventor contemplates compositions and methods for treating MAS comprising surfactant or buffer solution with the same pH a pharmaceutical formulation of surfactant in combination with any of the treatments discussed supra, such as a composition or method comprising an n-Acetylcysteine composition, a DNAse, and/or an antiprotease compound as described herein. Further, the inventor contemplates a treatment comprising a buffer solution with low pH for neutralizing meconium's digestive enzymes.

E. Treatment Protocols.

In some embodiments, the following treatment protocols are utilized.

1) Recommended Treatment Methods in the Delivery Room:

a. Thin Meconium:
i) The infant's oral- and nasopharynx should be suctioned by the obstetrician prior to delivery of the shoulders;
ii) In a clinically well-appearing newborn, visualization of the larynx and intubation should not be necessary;
iii) In a depressed newborn, intubate and suction first, then proceed with the resuscitation; and
iv) Administer pharmaceutical formulation.

b. Thick Meconium:
i) The infant's oral- and nasopharynx should be suctioned by the obstetrician prior to delivery of the shoulders;
ii) Following suctioning of the oral- and nasopharynx by the obstetrician, the infant's oral- and nasopharynx should be immediately suctioned by the pediatrician followed by endotracheal intubation and suctioning of any meconium that is present below the cords. In a clinically well-appearing, vigorously crying newborn without meconium at the level of the vocal cords, intubation may not be necessary;
iii) Visualize the cords via direct laryngoscopy and remove as much of the meconium from below the cords as possible. Do not apply suction to the tube by mouth. Use an adapter connecting the endotracheal tube directly to wall suction, with the pressure set at 40 to 60 TORR. Repeat the intubation as often as necessary to clear the lower airway of meconium, even if the infant has cried;
iv) Following suctioning, ventilate the infant as necessary;
v) Administer pharmaceutical formulation;
vi) Keep the infant warm and dry to prevent hypothermia and shunting.

Continue to monitor the infant's heart and respiratory rates;
vii) After the infant has been stable for a least five minutes, the stomach can be aspirated to remove as much of the meconium-stained fluid as possible; and
viii) If warranted by the clinical history (fetal distress, depressed infant, etc.), intubation should be performed even if meconium is not seen on the cords.
   2) Treatment in the Nursery:

a. The infant should be monitored and observed carefully for signs of respiratory distress, i.e., cyanosis, tachypnea, retractions, and grunting.

b. Arterial blood gases and pH should be monitored for evidence of either metabolic or respiratory acidosis.

c. Obtain a chest x-ray to rule out air leak (pneumothorax, pneumomediastinum, or pneumopericardium), secondary to air trapping from ball-valve obstruction.

d. An infant with a history of meconium aspiration who develops respiratory distress should be placed in a hood to maintain O2 saturations greater or equal to 99% to prevent episodes of hypoxia and shunting.

e. Postural drainage should be done as clinically indicated.

f. Consider intubation and suctioning below the cords in the nursery, since meconium can be removed from the upper airways even after the infant has initiated spontaneous respirations.

g. If the infant experiences persistent respiratory distress after one-half hour of life, antibiotics should be started after first obtaining blood, tracheal aspirate, and CSF cultures. Urine, for Group B Strep Latex, should also be obtained, but antibiotics should not be withheld while waiting for urine.

h. Monitor the infant for pulmonary hypertension with evidence of right-to-left shunting (See standard protocols for treatment of Pulmonary Hypertension).

i. Administer pharmaceutical preparation as necessary.

All publications and patents mentioned in the above specification are herein incorporated by reference in their entirety. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in medicine, neonatalology, biochemistry, chemistry, molecular biology, immunology or related fields are intended to be within the scope of the following claims.

What is claimed is:

1. A method of treating meconium aspiration syndrome, comprising,
a) providing,
i) a pharmaceutical formulation comprising therapeutically effective amounts of at least one compound selected from the group consisting of a meconium enzyme inhibitor compound, a meconium liquefying compound, and a meconium antiinflammatory compound; and
ii) a subject in need of prophylactic treatment for meconium aspiration syndrome; and
b) administering to said subject a therapeutically effective amount of said pharmaceutical formulation, wherein symptoms of meconium aspiration syndrome are reduced.

2. The method of claim 1, wherein a meconium enzyme inhibitor compound reduces activity of meconium enzymes or reduces activity of enzymes activated by contact with meconium.

3. The method of claim 2, wherein a meconium enzyme inhibitor compound is selected from the group consisting of antiprotease family of drugs, a buffer solution comprising a pH outside the optimal pH for pancreatic/intestinal digestive enzymes.

4. The method of claim 1, wherein a meconium liquefying compound reduces viscosity and/or thickness of meconium and is selected from the group consisting of N-acetylcysteine and DNAse.

5. The method of claim 1, wherein a meconium antiinflammatory compound is selected from a group consisting of antiprotease compounds.

6. The method of claim 5, wherein said antiprotease compound is selected from the group consisting of Aprotinin (Trasylol); Gabexate mesilate (Foy); Camostate (Foy 305); Nafamostate mesilate (FUT 175); Sepimostate (FUT 187); Ulinostatin; E 3123; THL (lipase inhibitor); C1 INH (C1-esterase inhibitor); CaNa2 (phospholipase A2 inhibitor); Procaine (phospholipase A2 inhibitor); and Lexipafant (PAF inhibitor) ONO 3307.

7. The method of claim 1, further comprising providing an artificial surfactant and administrating said surfactant in combination with said pharmaceutical preparation.

8. The method of claim 1 wherein said pharmaceutical preparation is administered by a method selected from the group consisting of intratracheal administration, amnio-infusion, oral administration, instillation, lavage, spraying, vaporizing, misting, nebulizing, systemical administration, intravenous administration, and intramuscular administration.

9. The method of claim 1 wherein said administering is oral with subsequent delivery of the active ingredients to the lungs or bloodstream for delivery of the active ingredient to the tracheal-bronchial tree.

10. The method of claim 1, wherein said administration of said pharmaceutical formulation reduces the severity of a symptom in the fetus or infant selected from the group consisting of airway obstruction, surfactant dysfunction, and chemical pneumonitis.

11. The method of claim 1, wherein said subject is a human.

12. A method of treating meconium aspiration syndrome, comprising,
a) providing,
i) a pharmaceutical formulation comprising a therapeutically effective amount of acetylcysteine; and
ii) a subject in need of prophylactic treatment for meconium aspiration syndrome; and
b) administering to said subject a therapeutically effective amount of said pharmaceutical formulation.

13. The method of claim 12, wherein said subject in need of prophylactic treatment for meconium aspiration syndrome by the presence of one or more indicators selected from the group consisting of meconium-stained amniotic fluid, intrauterine distress, placental insufficiency, maternal hypertension, preeclampsia, oligohydramnios, decreased amniotic fluid volume, maternal drug abuse, maternal tobacco use, and maternal cocaine abuse.

14. The method of claim 12, wherein said therapeutically effective amount reduces the toxicity of meconium.

15. The method of claim 12, further comprises administering a therapeutically effective amount of DNAse or an antiprotease compound or a combination thereof.

16. The method of claim 15, wherein said DNAse is human deoxyribonuclease I (rhDNase) or Pulmozyme® (dornase alfa).

17. A pharmaceutical preparation comprising therapeutically effective amounts of a meconium liquefying component and at least one antiprotease compound.

18. The pharmaceutical preparation of claim 17, wherein said meconium liquefying component is selected from the group consisting of DNase and N-Acetylcysteine or a combination thereof.

19. The pharmaceutical preparation of claim 17, wherein said at least one antiprotease compound is selected from the group consisting of Aprotinin (Trasylol), Gabexate mesilate (Foy), Camostate (Foy 305), Nafamostate mesilate (FUT 175), Sepimostate (FUT 187), Ulinostatin, E 3123, THL (lipase inhibitor), ONO5046 (PMN-elastase inhibitor), C1 INH (C1-esterase inhibitor), AT3 (antithrombin III inhibitor), CaNa2 (phospholipase A2 inhibitor), Procaine (phospholipase A2 inhibitor), Lexipafant (PAF inhibitor) ONO 3307.

20. A kit for therapeutic use comprising therapeutically effective amounts of a first component, wherein said first component is meconium liquefying substance and a second component, wherein said second component is at least one antiprotease compound, wherein said first and second components are provided in therapeutically effective amounts.
